(19) United States
(12) Patent Application Publication
(10) Pub. No.: US 2005/0065494 A1
(43) Pub. Date: Mar. 24, 2005

(54) Disposable feminine sanitary panty

(76) Inventor: Ava Harriott, Montreal (CA)

Correspondence Address: Mi La Shvartsman, P.O.Box 48652, CSP Outremont, Quebec, H2V 4T9, CA

(21) Appl. No.: 10/896,764

(22) Filed: Jun. 28, 2004

Related U.S. Application Data
(63) Continuation of application No. 08/360,254, filed on Dec. 20, 1994, now abandoned.

(51) Int. Cl. A61F 13/15; A61F 13/20

(52) U.S. Cl. 604/396

(57) ABSTRACT

The present invention relates to a disposable sanitary feminine panty comprising in combination an outer impermeable layer means, inner absorbent layer means comprising relatively thick absorbent layer, the inner absorbent layer being substantially coextensive with the outer impermeable layer means. Also, an extra pad absorbent means comprising a thick absorbent layer, the extra pad absorbent means placed substantially in the crotch area of the panty providing a relatively highly absorbent layer, an additional impermeable layer means disposed between the inner absorbent layer means and the extra pad absorbent means, an elastic waistband means for securing the sanitary panty to a user, and a leg band means through which the user's legs extend. The leg band means include elastic material to facilitate fitting on the user's body, and the elastic material is extending only in the areas adjacent to the wearer's hips to provide comfortable use of the panty. The extra pad absorbent means is permanently attached to the panty.

This application is a continuing application of Ser. No. 08/360,254 filed Dec. 20, 1994, now abandoned.

The present invention relates to disposable garment, and more particularly to a disposable panty designed for females having heavy flow during their menstrual periods.

There is known U.S. Pat. No. 4,743,239 related to a disposable brief having an area of relatively thin absorbent material, and an area of relatively thick absorbent material This brief is further comprising an exterior moisture barrier and an interior area having two different absorbent materials.

There is also known U.S. Pat. No. 5,098,419 related to an undergarment to be worn by incontinent persons comprising an upper portion made of a knitted fabric, and a lower crotch portion made of a woven, non-woven or knitted fabric. The crotch portion is coated with a modified polyester urethane polymer which prevents passage of body fluids.

At the present time there are known generally two types of disposable feminine products for use during menstruation:
sanitary pads;
tampons.

However, it is known that for a number of menstruating women, particularly those with heavy flow none of the mentioned above products are completely reliable, since its absorbent capacity is limited.

The sanitary feminine panty of the present invention overcomes the limitations and problems of the prior art products, would appeal to many menstrual women, particularly those with heavy flow, and would fulfil a need for a disposable panty that would have a pad or napkin as an integral part of its design.

The sanitary feminine panty would absorb leakage and prevent embarrassing stains, a stay-dry lining would prevent any leakage from reaching under/outer garments or bed clothes when worn at night.

The sanitary feminine panty according to the present invention would offer a no-mess, no-fuss, convenient way of dealing with leakage. The women would simply discard the sanitary panty and use a fresh pair. It would also eliminate the need to wash undergarment, which in the most of cases will be stained when conventional pad or tampon was used. The saving of time on having to wash and soak soiled clothes and undergarment and the damage to such items when badly stained would make the sanitary feminine panty appealing to many women, especially those who have heavy periods and cannot find a solution to the problem using existing products.

### SUMMARY OF THE INVENTION

Broadly, the present invention comprises a disposable sanitary feminine panty comprising in combination:
an outer impermeable layer means,
inner absorbent layer means comprising relatively thick absorbent layer, said inner absorbent layer is substantially coextensive with the outer impermeable layer means;
extra pad absorbent means comprising a thick absorbent layer, said extra pad absorbent means is placed substantially in a crotch area of said panty for providing a relatively highly absorbent layer;
an additional impermeable layer means disposed between said inner absorbent layer means and said extra pad absorbent means;
an elastic waistband means for securing the sanitary panty to a user; and
a leg band means through which the user's legs extend.

In another embodiment, said leg band means includes elastic material to facilitate fitting on the user's body, and said elastic material is extending only in the areas adjacent to the wearer's hips to provide comfortable use of said panty. Said extra pad absorbent means is permanently attached to said panty, and said additional impermeable layer means is also permanently attached to said panty.

In yet another embodiment, said outer impermeable layer is adapted to facilitate and promote evaporation from the user's body, and said additional impermeable layer is also adapted to facilitate and promote evaporation from the user's body.

### BRIEF DESCRIPTION OF DRAWINGS

FIG. 1 is a perspective view of sanitary feminine panty embodying the present invention.

FIG. 2 is a cross-section view taken along lines A-A of FIG. 1.

### DESCRIPTION OF THE PREFERRED EMBODIMENT

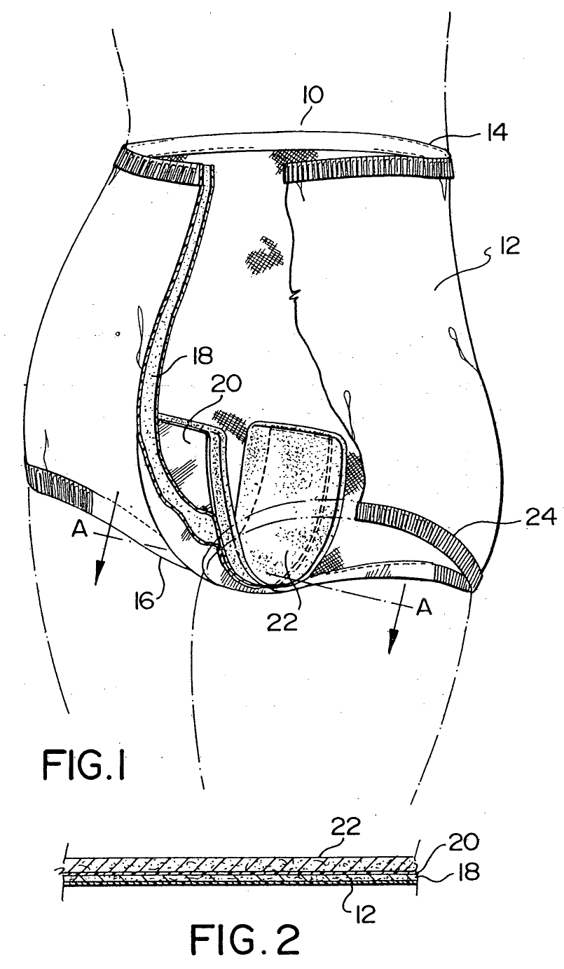

Referring now to drawings, FIG. 1 shows a perspective view of a sanitary feminine panty according to the present invention. The sanitary panty 10 is disposable and is adapted to be put and removed by the user in the conventional manner of a panty. The disposable sanitary feminine panty 10 includes an outer, generally moisture-impermeable layer or shell 12, which is made of same appropriate type of plastic, such as polyethylene, or the like. This outer waterproof shell 12 or stay-dry lining is intended too stop leakage of liquid, but in the same time is breathable to facilitate and promote evaporation of the woman's body wearing it. The panty includes an elastic waistband 14 and leg openings 16. An elastic band portions 24 are provided only near the hip areas of the wearer to facilitate comfortable wearing of the sanitary panty and to avoid any unnecessary frictions between the legs when in use. Adjacent to the outer impermeable layer 12, there is a relatively thick inner absorbent layer 18 which is continuous and extends over the total area of the sanitary panty. In the crotch area of panty 10 there is also provided an additional moisture impermeable or waterproof lining 20, which is disposed between said inner absorbent lining 18 and an additional extra pad 22. This additional extra pad 22 is made of relatively thick absorbent material and in combination with waterproof lining 20, inner absorbent layer 18 and outer waterproof shell 12 provides very secure means to prevent any leakage during menstrual period (see FIG. 2). The structure of the sanitary feminine panty 10 allows also to prevent infection by keeping the moisture away from direct contact with the body of the wearer. Preferably but not necessary, the additional extra pad 22 is permanently attached or stitched to the panty 10 together with the waterproof lining 20. The inner absorbent layer 18 and additional pad 22 are preferably made of a cotton or similar absorbent material. Even though the sanitary panty is in general intended to be used during menstrual flow, it also my be useful to retain urine or any other liquid or semi-liquid materials, thus this undergarment can accommodate incontinence adults. The total absorbency of the garment is not challenged by the amount of menstrual, or liquid, or semi-liquid fluids, since it has a large amount of absorbency as the menstrual fluid may flow, either in front, back or crotch area depending on the body position of the wearer, particularly when sleeping.

It is an important advantage of the present invention to have a higher amount of absorbency in each of these areas in order to always effectively absorb the moisture while exudating. This high absorbency also makes the panty suitable for incontinence use. The sanitary feminine panty of the present invention is lightweight, absorbent and provides optimum protection against leakage and stains. It can be produced in scented and unscented versions and can be made of various sizes and colours. The entire panty is covered on the outside with a stay-dry lining that would prevent any staining of outer garment or bed clothing when worn at night. It assures a good fit, elasticity, breathability and discreetness of a well fitting undergarment.

Because of its design, the sanitary feminine panty would serve its intended purpose well, and the wearer would not have to be concerned about becoming embarrassed in case of an accident. Stains would be prevented, therefore, the garment could be saved since often blood stains cannot be removed. Thus, the sanitary panty according to the present invention would give a woman protection, confidence and freedom.

While the principles of the invention have been made clear in illustrative embodiments, there will be immediately obvious to those skilled in the art many modifications of structure, arrangements, proportions, the elements, materials, and components used in the practice of the invention, and otherwise, which are particularly adapted for specific environments and operative requirements without departing from those principles. The appended claims are intended to cover and embrace any and all such modifications, within the limits only of the true spirit and scope of the invention. This specification and the appended claims have been prepared in accordance with the applicable patent laws and the rules promulgated under the authority thereof.

1. A disposable sanitary feminine panty comprising in combination:
an outer impermeable panty layer, said outer panty layer having a unitary, continuous perforation-free configuration;
an inner absorbent panty layer comprising substantially thick absorbent layer, said inner absorbent panty layer is placed coextensive with the outer impermeable panty layer
an extra pad comprising a substantially thick absorbent layer, said extra pad is placed over said inner absorbent layer in a crotch area of said panty to providing an additional absorbent layer;
an additional impermeable layer disposed between said inner absorbent layer and said extra pad, wherein said additional impermeable layer is provided to stop a flow of a fluid discharge coming in a direct contact with said extra pad, and wherein all excess flow of said fluid discharge leaking beyond an outer perimeter of said additional impermeable layer is adapted to be stopped by said inner absorbent panty layer disposed below said additional impermeable layer;
an elastic waistband for securing the sanitary panty to a user; and
a leg openings through which the user's legs extend, said leg openings are provided with leg bands, said leg bands are formed from an elastic material to facilitate fitting on the user's body, wherein said elastic material is extending only in the areas adjacent to the user's hips to provide comfortable use of said panty.

2. Sanitary feminine panty according to claim 1, wherein said extra pad is permanently attached to said panty.

3. Sanitary feminine panty according to claim 2, wherein said additional impermeable layer is also permanently attached to said panty.

4. Sanitary feminine panty according to claim 1, wherein said outer impermeable layer adapted to facilitate and promote evaporation from the user's body.

5. Sanitary feminine panty according to claim 1, wherein said additional impermeable layer is also adapted to facilitate and promote evaporation from the user's body.
